(19) United States
(12) Patent Application Publication
(10) Pub. No.: US 2023/0330187 A1
(43) Pub. Date: Oct. 19, 2023

(54) USE OF GHRELIN OR GHRELIN VARIANT FOR INCREASING DOSE INTENSITY OF CHEMOTHERAPEUTIC AGENTS IN PATIENTS WITH CANCER

(71) Applicant: OXEIA BIOPHARMACEUTICALS, INC., San Diego, CA (US)

(72) Inventors: Tony REID, San Diego, CA (US); Kartik SHAH, San Diego, CA (US)

(21) Appl. No.: 18/052,885

(22) Filed: Nov. 4, 2022

Related U.S. Application Data
(63) Continuation of application No. PCT/US2021/031172, filed on May 6, 2021.
(60) Provisional application No. 63/020,911, filed on May 6, 2020.
(60) Provisional application No. 63/020,920, filed on May 6, 2020.

(51) Int. Cl. A61K 38/22; A61K 45/06; A61P 35/00; A61K 31/33

(52) U.S. Cl. CPC A61K 38/22; A61K 45/06; A61P 35/00; A61K 31/33

(57) ABSTRACT

The disclosure describes the use of compositions comprising ghrelin or ghrelin variant to improve dose intensity, dose frequency, duration of use, reduced toxicity, and/or patient compliance of chemotherapeutic agents.

### FIELD OF THE DISCLOSURE

The disclosure is directed to the use of compositions comprising ghrelin or ghrelin variant to improve dose intensity, dose frequency, duration of use, reduced toxicity, and/or patient compliance of chemotherapeutic agents.

### BACKGROUND

Chemotherapeutic agents are an important aspect of cancer treatment. However, many cancer chemotherapeutic agents produce toxicity, even at usual therapeutic doses, that may cause significant side effects for the patients. In some cases, the toxicity and/or side effects are severe enough for chemotherapy treatment to be reduced or discontinued in the patient.

Methods and compositions for reducing toxicity and improving patient compliance with chemotherapeutic treatment are needed.

### SUMMARY

The disclosure provides compositions and methods for increasing dose intensity, e.g., dose frequency, dose density, dosage amount, and/or treatment duration of a chemotherapeutic agent in a subject in need thereof by administering ghrelin or a ghrelin variant and the chemotherapeutic agent to the subject.

In one aspect is provided a method for increasing survival in a subject having cancer, the method comprising administering to the subject ghrelin or a ghrelin variant, and a chemotherapeutic agent, thereby increasing survival or progression-free survival of the subject.

In one aspect is provided a method for increasing dose intensity of a chemotherapeutic agent in a subject having cancer, the method comprising administering to the subject ghrelin or a ghrelin variant, and the chemotherapeutic agent such that the dose intensity of the chemotherapeutic agent is increased.

In one aspect is provided a method for increasing dose frequency of a chemotherapeutic agent in a subject having cancer, the method comprising administering to the subject ghrelin or a ghrelin variant, and the chemotherapeutic agent such that the dose frequency of the chemotherapeutic agent is increased.

In one aspect is provided a method for increasing duration of treatment with a chemotherapeutic agent in a subject having cancer, the method comprising administering to the subject ghrelin or a ghrelin variant, and the chemotherapeutic agent such that the duration of treatment with the chemotherapeutic agent is increased.

In some embodiments, the chemotherapeutic agent is a kinase inhibitor, an anti-cancer antibody, an alkylating agent, an alkaloid, an antitumor antibiotic, an antimetabolite, or a topoisomerase inhibitor. In one embodiment, the chemotherapeutic agent is a platinum-based antineoplastic drug (platin) or a kinase inhibitor.

In one aspect is provided a method for increasing dose intensity, frequency, and/or duration of treatment with a chemotherapeutic agent in a subject having cancer, the method comprising administering to the subject ghrelin or a ghrelin variant and the chemotherapy agent, wherein the chemotherapy agent is a kinase inhibitor or a platinum-based antineoplastic drug (platin).

The method of any one of the preceding claims, wherein the ghrelin or ghrelin variant is administered after the subject has consumed a meal.

In one embodiment, the ghrelin or ghrelin variant is administered within about 2 hours of consumption of the meal. In one embodiment, the ghrelin or ghrelin variant is administered more than about 2 hours after consumption of the meal. In one embodiment, the meal is a low-fat meal. In one embodiment, the meal contains less than about 30% fat. In one embodiment, the meal is not a low-fat meal.

In one embodiment, the chemotherapeutic agent is administered after administration of ghrelin or ghrelin variant. In one embodiment, the chemotherapeutic agent is administered within about 2 hours of consumption of the meal. In one embodiment, the chemotherapeutic agent is administered within about 2 hours of ghrelin or ghrelin variant administration. In one embodiment, the chemotherapeutic agent is administered more than about 2 hours after consumption of the meal. In one embodiment, the chemotherapeutic agent is administered more than about 2 hours after ghrelin (or variant) administration. In particular, timing of administration of the chemotherapeutic agent will depend on the known or preferred timing for that agent. For example, the regorafenib package insert instructs that regorafenib is to be administered with a low-fat breakfast. In contrast, sorafenib is to be taken without food, at least 1 hour before or 2 hours after a meal.

In a preferred embodiment, ghrelin is administered. In one embodiment, one or more ghrelin variants is administered. In one embodiment, the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, LY444711, MK-0677, L-692,429, NNC 26-0703, EP 1572, Capromorelin (CP-424, 391-18, RQ-00000005), L-252,564, NN703, G-7203, S-37435, SM-130868, EX-1314, ulimorelin, macimorelin (acetate), anamorelin, ipamorelin, PF-5190457, AMX-213, tabimorelin, capromorelin, GHRP-6, or a combination thereof.

In one embodiment, the cancer is colorectal cancer, colon cancer, rectal cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, leukemia, acute leukemias, acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma or retinoblastoma. In a preferred embodiment, the cancer is colorectal cancer. In a more preferred embodiment, the colorectal cancer is metastatic colorectal cancer (mCRC). In one embodiment, the cancer is a gastrointestinal stromal tumor.

In some embodiments, the chemotherapy agent is a kinase inhibitor. In one embodiment, the kinase inhibitor is regorafenib or sorafenib. Preferably, the kinase inhibitor is regorafenib. In some embodiments, the regorafenib is administered for at least 10 consecutive days, at least 15 consecutive days, at least 18 consecutive days, or at least 20 consecutive days. In one embodiment, the regorafenib is administered for 21 days. In some embodiments, the regorafenib is administered for at least 10, 11, 12, 13, 14, 15, 16, 17, 18, 19, 20 or 21 days in a 28 day cycle. In some embodiments, the regorafenib is administered at a dose between 80 mg and 160 mg per day.

In some embodiments, the chemotherapy agent is a platin. In some embodiments, the platin is cisplatin, carboplatin, oxaliplatin, nedaplatin, triplatin tetranitrate, phenanthriplatin, picoplatin, or satraplatin. In a preferred embodiment, the platin is cisplatin.

In one embodiment, cognition in the subject is improved. In one embodiment, chemotherapy-related fatigue in the subject is reduced.

### DETAILED DESCRIPTION

### Definition of Terms

Unless defined otherwise, all technical and scientific terms used herein have the same meaning as commonly understood by one of skill in the art to which the inventions described herein belong. All publications, patents, and patent applications mentioned in this specification are hereby incorporated by reference to the same extent as if each individual publication, patent, or patent application was specifically and individually indicated to be incorporated by reference.

It must be noted that as used herein and in the appended claims, the singular forms “a”, “an”, and “the” include plural referents unless the context clearly dictates otherwise. Thus, for example, reference to “a compound” includes a plurality of compounds.

As used herein, the term “about” when used before a numerical designation, e.g., temperature, time, amount, concentration, and such other, including a range, indicates approximations which may vary by (+) or (−) 10%, 5% or 1%.

As used herein, the terms “comprising,” “including,” “such as,” and “for example” (or “e.g.”) are used in their open, non-limiting sense.

As used herein, the phrase “consisting essentially of” is a transitional phrase used in a claim to indicate that the following list of ingredients, parts or process steps must be present in the claimed composition, machine or process, but that the claim is open to unlisted ingredients, parts or process steps that do not materially affect the basic and novel properties of the invention.

“Subject” or “subject” refers to the animal (especially mammal) or human being treated.

A “subject group” or “subject group” or “patient population” has a sufficient number of subjects or subjects to provide a statistically significant average measurement of the relevant pharmacokinetic parameter.

As used herein, the term “administration” can be effected in one dose, continuously or intermittently or by several subdoses which in the aggregate provide for a single dose. Dosing can be conducted throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, vaginal, nasal administration, injection, topical application, sublingual, pulmonary, and by suppository.

The term “treat” and its grammatical variants (e.g., “to treat,” “treating,” and “treatment”) refer to administration of an active pharmaceutical ingredient to a subject with the purpose of ameliorating or reducing the incidence of one or more symptoms of a condition or disease state in the subject. Such symptoms may be chronic or acute; and such amelioration may be partial or complete.

The term “dose intensity” as used herein refers to the unit dose of chemotherapeutic agent administered per unit time.

The term “meal” as used herein refers to any intake of food, including but not limited to breakfast, lunch, brunch, dinner, and/or a snack.

The term “toxicity” or “chemotherapeutic toxicity” as used herein refers to the damaging effect an agent has on the subject. Common toxicities include, but are not limited to, cardiotoxicity, nephrotoxicity and hemorrhagic cystitis, hepatotoxicity, pulmonary toxicity, dermatologic toxicity, and gastrointestinal toxicity.

The term “therapeutic index” as used herein refers to the amount of a therapeutic agent that causes the therapeutic effect as compared to the amount that causes toxicity. In some embodiments, the therapeutic index is the ratio of the toxic dose to the effective dose of the therapeutic agent.

As used herein, the term “amino acid residue” refers to an amino acid formed upon chemical digestion (hydrolysis) of a polypeptide at its peptide linkages. Unless otherwise specified, the amino acid encompasses L-amino acid, including both natural amino acid and synthetic amino acid or the like as long as the desired functional property is retained by the polypeptide. NH2 refers to the free amino group present at the amino terminus of a polypeptide. COOH refers to the free carboxy group present at the carboxy terminus of a polypeptide. Standard polypeptide abbreviations for amino acid residues are as follows: A (Ala or Alanine); C (Cys or Cysteine); D (Asp or Aspartic Acid); E (Glu or Glutamic Acid); F (Phe or Phenylalanine); G (Gly or Glycine); H (His or Histidine); I (He or Isoleucine); K (Lys or Lysine); L (Leu or Leucine); M (Met or Methionine); N (Asn or Asparagine); P (Pro or Proline); Q (Gin or Glutamine); R (Arg or Arginine); S (Ser or Serine); T (Thr or Threonine); V (Val or Valine); W (Trp or Tryptophan); X (Xaa or Unknown or Other); Y (Tyr or Tyrosine); and Z (Glx/Gln/Glu or Glutamic Acid/Glutamine); and Dpr (2,3-diaminopropionic acid). All amino acid residue sequences represented herein by formula have a left-to-right orientation in the conventional direction of amino terminus to carboxy terminus. The phrase “amino acid residue” is broadly defined to include the naturally occurring and modified and non-naturally occurring amino acids. A dash at the beginning or end of an amino acid residue sequence indicates a peptide bond to a further sequence of one or more amino acid residues or a covalent bond to an amino-terminal group such as H2 or acetyl or to a carboxy-terminal group such as COOH.

As used herein, the term “amino acid derivatives” include, for example, alkyl-substituted tryptophan, β-naphthylalanine, naphthylalanine, 3,4-dihydrophenylalanine, and methylvaline. The amino acids and amino acid derivatives include both of L forms and D-forms.

The term “amino acid side chain” refers to any one of the twenty groups attached to the α-carbon in naturally occurring amino acids. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc.

As used herein, the term “ghrelin” is a polypeptide having 28 amino acid sequence as set forth in SEQ ID NO. 1, and can include the octanoyl acylation as described above. Human ghrelin is a polypeptide having the amino acid sequence as set forth in GenBank® Accession No. NP 057446 or Swiss-Prot Identifier GHRL HUMAN. Human ghrelin preprotein has 117 amino acids. This preprotein undergoes the following post-translational processing. The signal peptide (amino acids 1-23) is removed and the remaining 94 amino acids are cleaved by a protease to provide a mature 28 amino acid ghrelin (amino acids 24-51) or a mature 27 amino acid ghrelin (amino acids 24-50) and a mature 23 amino acid obestatin (amino acids 76-98). The 28 amino acid mature ghrelin peptide can be further modified at the serine at position 26 in the preprotein by either an O-octanoyl group or an O-decanoyl group. The obestatin mature peptide can be further modified at the lysine at position 98 of the preprotein by an amide group. An additional ghrelin preprotein is known, which lacks the glutamine at position 37 of the preprotein.

As used herein, the term “ghrelin variant” refers to any compound (e.g., peptides, small molecule drugs) having at least about 50% of a functional activity of ghrelin. The functional activity includes, without limitation, feeding regulation, nutrient absorption, gastrointestinal motility, energy homeostasis, anti-inflammatory regulation, suppression of inflammatory cytokines, activation of Gq/G11, accumulation of inositol phosphate, mobilization of calcium from intracellular stores, activation or deactivation of MAP kinases, FKB translocation, CRE driven gene transcription, reduction in reactive oxygen species (ROS), NAMPT enzyme activation, and/or binding of arrestin to ghrelin receptor. Examples of ghrelin variants are provided herein.

### Ghrelin and Ghrelin Variants

Ghrelin is a neuroendocrine hormone that acts as an endogenous ligand for growth hormone secretagogue receptor. It is a 28-amino acid and an endogenously produced peptide predominantly secreted by gastric mucosa. It has been referred to as the “hunger hormone,” due to its well-studied effects on appetite, but it also is believed to play a significant role in regulating the distribution and rate of use of energy.

Ghrelin and ghrelin variants are described, for example, in U.S. Pat. No. 9,119,832; and PCT Patent Publication Nos. WO 2015/120203; WO 2016/028826; WO 2016/048488; WO 2016/144978; and WO 2017/075535, each of which is incorporated herein by reference in its entirety.

In some embodiments, the ghrelin variants can be a sequence that includes any of a number of modifications to the wild type ghrelin sequence, which comprises a polypeptide having an amino acid sequence of Gly-Ser-Ser-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 1). Non-limiting examples of potential modifications include modifying the length (shorter or longer) of the sequences, modifying the chemistry of the amino acids, substituting one or more of the amino acids with another amino acid, a synthetic amino acid or otherwise rare or non-naturally occurring amino acid, introducing protecting groups at the N and/or C termini, etc. In some embodiments, the polypeptide is modified with one or more fatty acids. In some embodiments, the fatty acid is an octanoic acid. In some embodiments, the polypeptide is modified at serine at amino acid position 2 and/or serine at amino acid position 3 of SEQ ID NO. 1.

For example, in some embodiments, ghrelin or the ghrelin variants include C1-C20 acylation of the carboxyl group of one or both of the glutamic acid residues or of the C-terminus arginine group. In other embodiments, ghrelin or ghrelin variants include C1-C20 acylation of one or more of the hydroxyl groups of the serine residues. Yet, in other embodiments, ghrelin or ghrelin variants include replacing one or more of the L-amino acids with a D-amino acid. Every amino acid with the exception of glycine can occur in two isomeric forms, which are called L- and D-forms, analogous to left-handed and right-handed configurations. L-amino acids are the form commonly manufactured in cells and incorporated into proteins. As mentioned above, some ghrelin variants can have one or more of L-amino acids substituted with D-amino acids.

In some embodiments, one or more of the ghrelin variants listed above, can be specifically excluded.

In some embodiments, the ghrelin variant comprises or consists of a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO. 1 provided that in some embodiments such variants retain at least 50% of the activity of native ghrelin.

In some embodiments, the ghrelin variant is a ghrelin mimetic such as a compound which is one or more of RM-131 (Rhythm Pharmaceuticals, Boston, MA) (or BIM-28131 (Ipsen Group), Dln-101 (DiaLean Ltd., Israel), Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, and a combination thereof. In some embodiments, one or more of the ghrelin variants listed above can be specifically excluded. In some embodiments, those ghrelin variants which are a polypeptide have at least 80%, 85%, 89%, 90%, 91%, 92%, 93%, 94%, 95%, 96%, 97%, 98%, or 99% sequence identity to the amino acid sequence of one or more of the compounds described in the present disclosure. In some embodiments, ghrelin variants, which comprise short amino acid sequences, such as the RM-131 pentapeptide molecule, can have a substitution of one of its amino acids, for example, a conservative or other type of substitution as described herein with a natural or non-natural amino acids, as well as isomers of the same. Other chemical modifications also are contemplated, such as those described elsewhere herein (e.g., protecting groups, octanoylation, acylation, etc.). In some embodiments, one or more of the substitution listed above, can be specifically excluded. [0012] In some embodiments, the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethyl amide, isolated ghrelin splice variant-like compound, ghrelin splice variant, growth hormone secretagogue receptor GHS-R 1a ligand, LY444711, LY426410, hexarelin/examorelin, growth hormone releasing hexapeptide-1 (GHRP-I), GHRP-2, GHRP-6 (SK&F-110679), ipamorelin, MK-0677, NN703, capromorelin, CP 464709, pralmorelin, macimorelin (acetate), anamorelin, relamorelin, ulimorelin, ipamorelin, tabimorelin, ibutamoren, G7039, G7134, G7203, G-7203, G7502, SM-130686, RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291, MK-0677, L-692,429, EP 1572, L-252,564, NN703, S-37435, EX-1314, PF-5190457, AMX-213, and a combination thereof. In some embodiments, one or more of the ghrelin variants listed above, can be specifically excluded.

In some embodiments, the ghrelin variant comprises a polypeptide comprising the sequence of Gly Ser Ser Phe Leu Ser Pro Glu His Gin Arg Val Gin Val Arg Pro Pro Lys Ala Pro His Val Val (SEQ ID No. 2). In some embodiments, the ghrelin variant comprises a polypeptide comprising the sequence of Gly Ser Xaa Phe Leu Ser Pro Glu His Gin Arg Val Gin Val Arg Pro Pro His Lys Ala Pro His Val Val (SEQ ID No. 3), wherein the third position is a 2,3-diaminopropionic acid (Dpr), with the Dpr in the third position being optionally octanoylated. In some embodiments, the ghrelin variant comprises a polypeptide comprising the sequence of Gly Xaa Xaa Phe Leu Ser Pro Glu His Gin Arg Val Gin Val Arg Pro Pro His Lys Ala Pro His Val Val (SEQ ID No. 4), wherein the second and third position are 2,3-diaminopropionic acid (Dpr) residues, with the Dpr in the third position being optionally octanoylated. In some embodiments, the ghrelin variant comprises a polypeptide comprising the sequence of Gly Ser Ser Phe Leu Ser Pro Glu His Gin Arg Val Gin Val Arg Pro Pro His Lys Ala Pro His Val Val Pro Ala Leu Pro (SEQ ID No. 5). In some embodiments, the ghrelin variant comprises a polypeptide having at least 80%, 81%, 82%, 83%, 84%, 85%, 86%, 87%, 88%, 89%, 90%, 91%, 92%, 93%, 94%, 95%), 96%), 97%), 98%), or 99% sequence identity to the amino acid sequence of one or more of the compounds described in the present paragraph. In some embodiments, the ghrelin variant comprises a polypeptide comprising the sequence of Inp-D-2Nal-D-T-Thr-Lys-NH2 (SEQ ID No. 6). In some embodiments, one or more of the ghrelin variants listed above, can be specifically excluded.

In some embodiments, the ghrelin variant is one or more of LY444711 (2(R)-(2-Amino-2-methylpropanamido)-N-[1-[1 (R)-(4-methoxyphenyl)-1-methyl-2-oxo-2-(1-pyrrolidinypethyl]-1H-imidazol-4-yl]-5-phenylpentanamide dihydrochloride, C32H44CI2N604; Ely Lilly), MK-0677 (2-Amino-N-[2-benzyloxy-(1R)-[1-(methanesulfonyl)spiro[indoline-3,4′-piperidin]-1′-ylcarbonyl]ethyl]isobutyramide methanesulfonate, C28H40N4O8S2; Merck & Co., Inc.), L-692,429 (Merck Research Laboratories), Tabimorelin (NNC 26-0703, NN703; N-[5-Amino-5-methyl-2(E)-hexenoyl]-N-methyl-3-(2-naphthyl)-D-alanyl-N-methyl-D-phenylalanine methylamide, C32H40N4O3; Novo Nordisk), Capromorelin (CP-424,391-18; RQ-00000005; CP-424391; 2-Amino-N-[243a(R)-benzyl-2-methyl-3-oxo-3,3a,4,5,6,7-hexahydro-2H-pyrazolo[4,3-c]pyridin-5-yl]-1(R)-(benzyloxymethyl)-2-oxoethyllisobutyramide, C28H35N5O4; Pfizer and RaQualia, Japan), L-252,564 (Merck), G-7203, S-37435 (N-[1(R)-[N-(3-Amino-2-hydroxypropyl)carbamoyl]-2-naphthylethyl]-4-(4-oxo-2,3,4,5-tetrahydro-1,5-benzothiazepin-5-yl)butyramide hydrochloride, C29H35CIN4O4S; Kaken/Molecular Research Institute), SM-130868 ((+)-(35)-3-(2-chlorophenyl)-1-[2-(diethylamino)ethyl]-3-hydroxo-2-oxo-4-(trifluoromethyl)indoline-6-carboxamide, C22H23CIF3N303, Sumitomo), EX-1314 (Carbamic acid, (2-amino-2-oxoethyl)methyl-, (3-((1S)-1-((2-amino-2-methyl-1-oxopropyl)amino)-2-(phenylmethoxy)ethyl)-1,2,4-triazolo(4,3-a)pyridin-5-yl)methyl ester, monohydrochloride, C24H31N7O5 HCl; Elixir Pharmaceuticals), ulimorelin((2R,5S,8R,11R)-5-Cyclopropyl-11-(4-fluorobenzyl)-2,7,8-trimethyl-4,5,7,8,10,11,13,14,15,16-decahydro-2H-1,4,7,10,13-benzoxatetraazacyclooctadecine-6,9,12(3H)-tri one, C30H39FN404; Tranzyme Pharma, Inc., Ocera Therapeutics, Lyric Pharmaceuticals, Inc.), macimorelin (acetate) (EP 1572; 2-Amino-N-[(2R)-1-[[(1R)-1-formamido-2-(1H-indol-3-yl)ethyl]amino]-3-1H-indol-3-yl)-1-oxopropan-2-yl]-2-methylpropanamide, C26H30N6O3, Sterna Zentaris Inc.), anamorelin (HCl) (2-amino-N-((R)-1-((R)-3-benzyl-3-(1,2,2-trimethylhydrazinecarbonyl)piperidin-1-yl)-3-(1H-indol-3-yl)-1-oxopropan-2-yl)-2-methylpropanamide hydrochloride, C31H43CIN6O3; Helsinn Group), ipamorelin (2-Methylalanyl-L-histidyl-3-(2-naphthyl)-D-alanyl-D-phenylalanyl-L-lysinamide,C38H49N9O5), PF-5190457, GHRP-6 (L-histidyl-D-tryptophyl-L-alanyl-L-tryptophyl-D-phenylalanyl-L-Lysinamide), AMX-213 (Ammunix), and a combination thereof. In some embodiments, one or more of the ghrelin variants listed above, can be specifically excluded.

In some embodiments, the ghrelin variant is one or more of LY426410 (2-Methylalanyl-N-[1-[1 (R)-(4-methoxyphenyl)-2-(4-methyl-1-piperidinyl)-2-oxoethyl]-1H-imidazol-4-yl]-O-benzyl-D-serinamide; 2-Amino-N-[1 (R)-(benzyloxymethyl)-2-[1-[1 (R)-(4-methoxyphenyl)-2-(4-methyl-1-piperidinyl)-2-oxoethyl]-1H-imidazol-4-ylamino]-2-oxoe), hexarelin/examorelin ((2S)-6-amino-2-[[(2R)-2-[[(2S)-2-[[(2S)-2-[[(2R)-2-[[(2S)-2-amino-3-(1H-imidazol-5-yl)propanoyl]amino]-3-(2-methyl-1H-indol-3-yl)propanoyl]amino]propanoyl]amino]-3-(1H-indol-3-yl)propanoyl]amino]-3-phenylpropanoyl]amino]hexanamide), growth hormone releasing hexapeptide-1 (GHRP-I), GHRP-2, GHRP-6 (SK&F-1 10679), CP 464709 (2-amino-N-[(2R)-1-[(3aR)-3-oxo-3a-(pyridin-2-ylmethyl)-2-(2,2,2-trifluoroethyl)-6,7-dihydro-4H-pyrazolo[4,3-c]pyridin-5-yl]-3-[(2,4-difluorophenyl)methoxy]-1-oxopropan-2-yl]-2-methylpropanamide), pralmorelin ((2S)-6-amino-2-[[(2S)-2-[[(2S)-2-[[(2S)-2-[[(2R)-2-[[(2R)-2-aminopropanoyl]amino]-3-naphthalen-2-ylpropanoyl]amino]propanoyl]amino]-3-(1H-indol-3-yl)propanoyl]amino]-3-phenylpropanoyl]amino]hexanamide), relamorelin (4-[[(2S)-2-[[(2R)-2-[[(2R)-3-(1-benzothiophen-3-yl)-2-(piperidine-3-carbonylamino)propanoyl]amino]-3-(1H-indol-3-yl)propanoyl]amino]-3-phenylpropanoyl]amino]piperidine-4-carboxamide), ulimorelin, ((7R,1OR,13S,16R)-13-cyclopropyl-7-[(4-fluorophenyl)methyl]-10,11,16-trimethyl-17-oxa-5,8,11,14-tetrazabicyclo[16.4.0]docosa-1(22),18,20-triene-6,9,12-trione), tabimorelin (N-[(2E)-5-amino-5-methylhex-enoyl]-N-methyl-3-(2-naphthyl)alanyl-N,Na-dimethyl-D-phenyl alaninamide; NN703; NNC 26-0703), ibutamoren (2-amino-2-methyl-N-[(2R)-1-(1-methylsulfonylspiro[2H-indole-3,4′-piperidine]- -yl)-1-oxo-3-phenylmethoxypropan-2-yl]propanamide), G7039 (Genentech), G7134 (Genentech), G7502 ([[5-(2-amino-6-oxo-3H-purin-9-yl)-3,4-dihydroxyoxolan-2-yl]methoxy-hydroxyphosphoryl] [3,4,5-trihydroxy-6-(hydroxymethyl)oxan-2-yl] hydrogen phosphate), Anamorelin/RC-1291 (2-amino-N-[(2R)-1-[(3R)-3-benzyl-3-[dim ethyl amino(methyl)carbamoyl]piperidin-1-yl]-3-(1H-indol-3-yl)-1-oxopropan-2-yl]-2-methylpropanamide), L-692429 (3-amino-3-methyl-N-[(3R)-2-oxo-1-[[4-[2-(2H-tetrazol-5-yl)phenyl]phenyl]methyl]-4,5-dihydro-3H-1-benzazepin-3-yl]butanamide), Pexiganan [INN]/L-692587 (Glycyl-L-isoleucylglycyl-L-lysyl-L-phenylalanyl-L-leucyl-L-lysyl-L-lysyl-L-alanyl-L-lysyl-L-lysyl-L-phenylalanylglycyl-L-lysyl-L-alanyl-L- phenylalanyl-L-valyl-L-lysyl-L-isoleucyl-L-leucyl-L-lysyl-L-lysinamide) (SEQ ID NO: 37), CHEMBL291200/L-739943 (3-amino-3-methyl-N-[(3R)-1-[[4-[2-[(methylcarbamoylamino)methyl]phenyl]phenyl]methyl]-2-oxo-4,5-dihydro-3H-1-benzazepin-3-yl]butanamide), L-163255 (2-amino-2-methyl-N-[(2R)-1-(1-methylsulfonylspiro[2H-indole-3,4′-piperidine]- -yl)-1-oxo-5-phenylpentan-2-yl]propanamide), L-163,540 (1-[2(R)-(2-amino-2-methylpropionylamino)-3-0H-indol-3-yl)propionyl]-3-benzylpiperidine-3(S)-carboxylic acid ethyl ester), L-163833, L-166446, EP-51389, NNC-26-0235 (3-(aminomethyl)benzoyl-2-naphthylalanyl-N-methylphenylalanyl-lysinamide, 3-(aminomethyl)benzoyl-2Nal-N-Me-Phe-Lys-NH2, NNC-26-0235), NNC-26-0323 (3-(aminomethyl)benzoyl-D-2Nal-N-Me-D-Phe-Lys-NH2), NNC-26-0610 (Novo Nordisk), NNC-26-0722, NNC-26-1089, NNC-26-1136, NNC-26-1137, NNC-26-1187, NNC-26-1291, EP 1572 (Aib-DTrp-DgTrp-CHO), S-37435 (N-[(2R)-1-[(3-amino-2-hydroxypropyl)amino]-3-naphthalen-2-yl-1-oxopropan-2-yl]-4-(1,1,4-trioxo-2,3-dihydro-1 {6},5-benzothiazepin-5-yl)butanamide), and a combination thereof. In some embodiments, one or more of the ghrelin variants listed above, can be specifically excluded.

In some embodiments, one or more of the ghrelin variants listed herein may be specifically excluded.

### Ghrelin or Ghrelin Variant to Improve Chemotherapy Compliance

This disclosure is directed to compositions and methods for increasing dose intensity, e.g., dose frequency, dose density, dosage amount, and/or treatment duration of a chemotherapeutic agent in a subject in need thereof by administering ghrelin and the chemotherapeutic agent to the subject. In one aspect, treatment with ghrelin or ghrelin variant in combination with the chemotherapeutic agent improves the therapeutic index of the drug (i.e., decreases the effective dose and/or increases the toxic dose).

Dose intensity is important to clinical outcomes in patients with potentially curable cancers, as increased dose intensity is correlated with improved clinical outcomes (e.g., disease control, survival). See, e.g., Lyman, J Natl Compr Canc Netw. 2009 January; 7(1):99-108; Gray et al., “Increasing the dose density of adjuvant chemotherapy by shortening intervals between courses or by sequential drug administration significantly reduces both disease recurrence and breast cancer mortality: An EBCTCG meta-analysis of 21,000 women in 16 randomised trials,” Presented at: 40^{th} Annual San Antonio Breast Cancer Symposium: 2017 Dec. 5-9, San Antonio, Texas; each of which is incorporated herein by reference in its entirety.

Toxicity of chemotherapeutic agents can affect patient compliance and dosing. For example, dosing of the chemotherapeutic agent may be reduced in response to toxicity in the patient. Patients may be hesitant or unwilling to take the chemotherapeutic agent, or be more likely to miss doses, in response to toxicity and side effects. In some cases, the chemotherapeutic agent may be discontinued earlier than recommended (e.g., after fewer than the recommended doses). The present invention is expected to address this issue by reducing toxicity in patients so as to improve compliance, e.g. with the recommended dosage schedule.

In some embodiments, treatment of the subject with ghrelin or ghrelin variant in addition to the chemotherapeutic agent results in improved patient compliance with the chemotherapeutic treatment, increased dose intensity of the chemotherapeutic agent, increased duration of treatment with the chemotherapeutic agent, increased dosage (e.g., daily dosage) of the chemotherapeutic agent, improved therapeutic index for the chemotherapeutic agent, reduced incidence of toxicity in the patient, and/or reduced side effects in the patient. In some embodiments, treatment of the subject with ghrelin or ghrelin variant in addition to the chemotherapeutic agent results in reduction in tumor size, increased survival, improved progression-free survival, and/or improved cognition. Improvement may be relative to, for example and without limitation, the patient prior to treatment with ghrelin (or variant), relative to a patient population or expected response, etc.

In one aspect is provided a method for increasing survival in a subject having cancer, the method comprising administering to the subject ghrelin or a ghrelin variant, and a chemotherapeutic agent, thereby increasing survival or progression-free survival of the subject. In some embodiments, progression free survival is increased by at least about 10%, 20%, 30%, 40%, 50%, 60%, 70%, 80%, 90%, 100%, or more.

In one aspect is provided a method for increasing dose intensity of a chemotherapeutic agent in a subject having cancer, the method comprising administering to the subject ghrelin or a ghrelin variant, and the chemotherapeutic agent such that the dose intensity of the chemotherapeutic agent is increased. In some embodiments, the dose intensity of the chemotherapeutic agent is increased by at least about 10%, 20%, 30%, 40%, 50%, 60%, 70%, 80%, 90%, 100%, or more.

In one aspect is provided a method for increasing dose frequency of a chemotherapeutic agent in a subject having cancer, the method comprising administering to the subject ghrelin or a ghrelin variant, and the chemotherapeutic agent such that the dose frequency of the chemotherapeutic agent is increased. In some embodiments, the dose frequency of the chemotherapeutic agent is increased by at least about 10%, 20%, 30%, 40%, 50%, 60%, 70%, 80%, 90%, 100%, or more.

In one aspect is provided a method for increasing duration of treatment with a chemotherapeutic agent in a subject having cancer, the method comprising administering to the subject ghrelin or a ghrelin variant, and the chemotherapeutic agent such that the duration of treatment with the chemotherapeutic agent is increased. In some embodiments, the duration of treatment of the chemotherapeutic agent is increased by at least about 10%, 20%, 30%, 40%, 50%, 60%, 70%, 80%, 90%, 100%, or more.

In one aspect is provided a method for improving the therapeutic index of a chemotherapeutic agent in a subject having cancer, the method comprising administering to the subject ghrelin or a ghrelin variant, and the chemotherapeutic agent such that the therapeutic index of the chemotherapeutic agent is increased. In some embodiments, the therapeutic index of the chemotherapeutic agent is increased by at least about 10%, 20%, 30%, 40%, 50%, 60%, 70%, 80%, 90%, 100%, or more.

In one aspect is provided a method for increasing dose intensity, frequency, and/or duration of treatment with a chemotherapeutic agent in a subject having cancer, the method comprising administering to the subject ghrelin or a ghrelin variant and the chemotherapy agent, wherein the chemotherapy agent is a kinase inhibitor or a platinum-based antineoplastic drug (platin).

In a preferred embodiment, ghrelin is administered. In one embodiment, one or more ghrelin variants is administered. In one embodiment, the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, LY444711, MK-0677, L-692,429, NNC 26-0703, EP 1572, Capromorelin (CP-424, 391-18, RQ-00000005), L-252,564, NN703, G-7203, S-37435, SM-130868, EX-1314, ulimorelin, macimorelin (acetate), anamorelin, ipamorelin, PF-5190457, AMX-213, tabimorelin, capromorelin, GHRP-6, or a combination thereof.

In some embodiments, the chemotherapy agent is a kinase inhibitor. In one embodiment, the kinase inhibitor is regorafenib or sorafenib. Preferably, the kinase inhibitor is regorafenib. In some embodiments, the regorafenib is administered for least 7 consecutive days, least 8 consecutive days, least 9 consecutive days, at least 10 consecutive days, least 11 consecutive days, least 12 consecutive days, least 13 consecutive days, least 14 consecutive days, at least 15 consecutive days, least 16 consecutive days, least 17 consecutive days, at least 18 consecutive days, least 19 consecutive days, or at least 20 consecutive days. In one embodiment, the regorafenib is administered for 21 days. In some embodiments, the regorafenib is administered for at least 7, 8, 9, 10, 11, 12, 13, 14, 15, 16, 17, 18, 19, 20 or 21 days in a 28 day cycle.

In some embodiments, administration of ghrelin or ghrelin variant in combination with regorafenib increases the number of days that regorafenib is administered to the subject by at least 10%, 20%, 30%, 40%, 50%, 60%, 70%, 80%, 90%, 100%, or more. In some embodiments, administration of ghrelin or ghrelin variant in combination with regorafenib increases the number of 28-day cycles that regorafenib is administered to the subject by at least 1, 2, 3, 4, 5, 6, 7, 8, 9, 10, or more cycles.

In some embodiments, the regorafenib is administered at a dose between 80 mg and 160 mg per day. In one embodiment, the regorafenib is administered at a dose of 80 mg per day. In one embodiment, the regorafenib is administered at a dose of 120 mg per day. In one embodiment, the regorafenib is administered at a dose of 160 mg per day. In one embodiment, administration of ghrelin or ghrelin variant in combination with regorafenib increases the amount of regorafenib that can be administered to the patient.

### Chemotherapeutic Agents

The chemotherapeutic agent may be any such agent, which are well known in the art. For example and without limitation, the chemotherapeutic agent may be a kinase inhibitor, an anti-cancer antibody, an alkylating agent, an alkaloid, an antitumor antibiotic, an antimetabolite, or a topoisomerase inhibitor. In one embodiment, the chemotherapeutic agent is a platinum-based antineoplastic drug (platin) or a kinase inhibitor.

In some embodiments, the chemotherapy agent is a platin. In some embodiments, the platin is cisplatin, carboplatin, oxaliplatin, nedaplatin, triplatin tetranitrate, phenanthriplatin, picoplatin, or satraplatin. In a preferred embodiment, the platin is cisplatin.

In a preferred embodiment, the chemotherapeutic agent is a kinase inhibitor (e.g., a tyrosine kinase inhibitor). In one embodiment, the tyrosine kinase inhibitor is afatinib, axitinib, cetuximab, cobimetinib, crizotinib, entrectinib, fostamatinib, ibrutinib, lenvatinib, mubritinib, pazopanib, pegaptanib, ruxolitinib, SU6656, vandetanib, vemurafenib, imantinib, gefinitib, toceranib, Erlotinib, Lapatinib, Sunitinib, Sorafenib, Nilotinib, bosutinib, neratinib, vatalanib, desatinib, ponatinib, trastuzumab, Ranibizumab, or regorafenib. In a particularly preferred embodiment, the chemotherapeutic agent is regorafenib.

In some embodiments, one or more of the chemotherapeutic agents listed herein may be specifically excluded.

### Cancer Types

In one embodiment, the cancer is colorectal cancer, colon cancer, rectal cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, leukemia, acute leukemias, acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma or retinoblastoma. In some embodiments, one or more of the cancers listed herein may be specifically excluded.

In a preferred embodiment, the cancer is colorectal cancer. In a more preferred embodiment, the colorectal cancer is metastatic colorectal cancer (mCRC).

In one embodiment, the cancer is a gastrointestinal stromal tumor.

### Patient Selection

In some embodiments, the methods as described herein comprise selecting a patient in need of ghrelin treatment in combination with a chemotherapeutic agent. In one embodiment, the patient is being treated or expected to be treated with a chemotherapeutic agent. In one embodiment, the patient is susceptible to toxicity of the chemotherapeutic agent. In one embodiment, the patient has one or more symptoms of toxicity to the chemotherapeutic agent.

In one aspect is provided a method for increasing dose intensity of a chemotherapeutic agent in a subject having cancer, the method comprising selecting a subject susceptible to toxicity due to the chemotherapeutic agent, and administering to the subject ghrelin or a ghrelin variant and the chemotherapeutic agent such that the dose intensity of the chemotherapeutic agent is increased.

In one aspect is provided a method for increasing dose frequency of a chemotherapeutic agent in a subject having cancer, the method comprising administering to the subject ghrelin or a ghrelin variant, and the chemotherapeutic agent such that the dose frequency of the chemotherapeutic agent is increased.

In one aspect is provided a method for increasing duration of treatment with a chemotherapeutic agent in a subject having cancer, the method comprising administering to the subject ghrelin or a ghrelin variant, and the chemotherapeutic agent such that the duration of treatment with the chemotherapeutic agent is increased.

In one embodiment, the patient has colorectal cancer. In one embodiment, the patient has metastatic colorectal cancer. In one embodiment, the patient has metastatic colorectal cancer (CRC) and has been previously treated with fluoropyrimidine-, oxaliplatin- and irinotecan-based chemotherapy, an anti-VEGF therapy, and, if KRAS wild type, an anti-EGFR therapy.

In one embodiment, the patient has a gastrointestinal stromal tumor. In one embodiment, the patient has locally advanced, unresectable or metastatic gastrointestinal stromal tumor (GIST). In one embodiment, the patient has locally advanced, unresectable or metastatic gastrointestinal stromal tumor (GIST) and has been previously treated with imatinib mesylate and sunitinib malate.

### Dosing of Ghrelin or Ghrelin Variant and the Chemotherapeutic Agent

In general, the dose of the chemotherapeutic agent in the methods described herein is the same or similar to that which is known in the art, for example as described in the prescribing information (e.g., package insert) for the chemotherapeutic agent. However, toxicity often forces the clinician or the patient to reduce the amount of chemotherapeutic agent administered (e.g., amount per dose, amount per day, total number of doses over time, etc.), or to discontinue administration of the chemotherapeutic agent altogether. Preferably, administration of ghrelin or ghrelin variant in combination with the chemotherapeutic agent results in reduced toxicity so as to avoid such reduction or discontinuation. In one embodiment, the chemotherapy agent is administered orally.

Ghrelin or ghrelin variant may be administered as described, for example, in U.S. Pat. No. 9,119,832; and PCT Patent Publication Nos. WO 2015/120203; WO 2016/028826; WO 2016/048488; WO 2016/144978; and WO 2017/075535, each of which is incorporated herein by reference in its entirety. In one embodiment, the ghrelin is administered by injection.

In some embodiments, the ghrelin or ghrelin variant is administered after the subject has consumed a meal. In some embodiments, the ghrelin or ghrelin variant is administered before the subject has consumed a meal.

In one embodiment, the meal is a low-fat meal. In one embodiment, the meal contains less than about 30% fat. In one embodiment, the meal is not a low-fat meal.

In one embodiment, the ghrelin or ghrelin variant is administered within about 2 hours after consumption of the meal. In one embodiment, the ghrelin or ghrelin variant is administered within about 1 hour after consumption of the meal. In one embodiment, the ghrelin or ghrelin variant is administered within about 45 minutes after consumption of the meal. In one embodiment, the ghrelin or ghrelin variant is administered within about 30 minutes after consumption of the meal. In one embodiment, the ghrelin or ghrelin variant is administered within about 15 minutes after consumption of the meal. In one embodiment, the ghrelin or ghrelin variant is administered within about 10 minutes after consumption of the meal. In one embodiment, the ghrelin or ghrelin variant is administered concurrently with or immediately after consumption of the meal.

In one embodiment, the ghrelin or ghrelin variant is administered more than about 30 minutes after consumption of the meal. In one embodiment, the ghrelin or ghrelin variant is administered more than about 1 hour after consumption of the meal. In one embodiment, the ghrelin or ghrelin variant is administered more than about 2 hours after consumption of the meal. In one embodiment, the ghrelin or ghrelin variant is administered more than about 3 hours after consumption of the meal.

In one embodiment, the chemotherapeutic agent is administered after administration of ghrelin or ghrelin variant. In one embodiment, the chemotherapeutic agent is administered within about 10 minutes, about 20 minutes, about 30 minutes, about 45 minutes, about 1 hour, or about 2 hours after consumption of the meal.

In one embodiment, the chemotherapeutic agent is administered within about 10 minutes, about 20 minutes, about 30 minutes, about 45 minutes, about 1 hour, or about 2 hours after ghrelin or ghrelin variant administration.

In one embodiment, the chemotherapeutic agent is administered more than about 2 hours after consumption of the meal. In one embodiment, the chemotherapeutic agent is administered more than about 3 hours after consumption of the meal. In one embodiment, the chemotherapeutic agent is administered more than about 2 hours after ghrelin administration. In one embodiment, the chemotherapeutic agent is administered more than about 3 hours after ghrelin administration.

In one embodiment, the ghrelin or ghrelin variant is administered within about 2 hours before consumption of the meal. In one embodiment, the ghrelin or ghrelin variant is administered within about 1 hour before consumption of the meal. In one embodiment, the ghrelin or ghrelin variant is administered within about 45 minutes before consumption of the meal. In one embodiment, the ghrelin or ghrelin variant is administered within about 30 minutes before consumption of the meal. In one embodiment, the ghrelin or ghrelin variant is administered within about 15 minutes before consumption of the meal. In one embodiment, the ghrelin or ghrelin variant is administered within about 10 minutes before consumption of the meal. In one embodiment, the ghrelin or ghrelin variant is administered immediately before consumption of the meal.

In one embodiment, the ghrelin or ghrelin variant is administered more than about 30 minutes before consumption of the meal. In one embodiment, the ghrelin or ghrelin variant is administered more than about 1 hour before consumption of the meal. In one embodiment, the ghrelin or ghrelin variant is administered more than about 2 hours before consumption of the meal. In one embodiment, the ghrelin or ghrelin variant is administered more than about 3 hours before consumption of the meal.

In one embodiment, the chemotherapeutic agent is administered before consumption of the meal. In one embodiment, the chemotherapeutic agent is administered within about 10 minutes, about 20 minutes, about 30 minutes, about 45 minutes, about 1 hour, or about 2 hours before consumption of the meal.

In one embodiment, the chemotherapeutic agent is administered before administration of ghrelin or ghrelin variant. In one embodiment, the chemotherapeutic agent is administered within about 10 minutes, about 20 minutes, about 30 minutes, about 45 minutes, about 1 hour, or about 2 hours before ghrelin or ghrelin variant administration.

In one embodiment, the chemotherapeutic agent is administered more than about 2 hours before consumption of the meal. In one embodiment, the chemotherapeutic agent is administered more than about 3 hours before consumption of the meal. In one embodiment, the chemotherapeutic agent is administered more than about 2 hours before ghrelin or ghrelin variant administration. In one embodiment, the chemotherapeutic agent is administered more than about 3 hours before ghrelin or ghrelin variant administration.

In particular, timing of administration of the chemotherapeutic agent will depend on the known or preferred timing for that agent. For example, the regorafenib package insert instructs that regorafenib is to be administered with a low-fat breakfast. In contrast, sorafenib is to be taken without food, at least 1 hour before or 2 hours after a meal.

In one aspect is provided a method for increasing dose intensity, frequency, and/or duration of treatment with a chemotherapeutic agent in a subject having cancer, the method comprising (a) administering to the subject ghrelin or a ghrelin variant within about 2 hours of consumption of a meal by the subject; and (b) administering to the subject the chemotherapy agent within about 2 hours of administration of ghrelin or ghrelin variant. In a preferred embodiment, the chemotherapy agent is a kinase inhibitor or a platinum-based antineoplastic drug (platin). In another preferred embodiment, the chemotherapy agent is cisplatin. In an especially preferred embodiment, the chemotherapy agent is regorafenib.

In one aspect is provided a method for increasing dose intensity, frequency, and/or duration of treatment with regorafenib in a subject having cancer, the method comprising (a) administering to the subject ghrelin or a ghrelin variant within about 2 hours after consumption of a meal by the subject; (b) administering to the subject regorafenib within about 2 hours after administration of ghrelin or ghrelin variant. In a preferred embodiment, the ghrelin or ghrelin variant and/or regorafenib is administered within about 30 minutes after consumption of the meal. In one embodiment, the cancer is colorectal cancer (CRC). In one embodiment, the cancer is metastatic CRC.

### EXAMPLE

### Example 1: Treatment of Colorectal Cancer Using Ghrelin and Regorafenib

Patients with metastatic colorectal cancer (mCRC) are administered ghrelin and regorafenib (STIVARGA™) in combination. Patients consume a low-fat breakfast, followed by ghrelin injection. Regorafenib (80 mg, 120 mg, or 160 mg) is orally administered within about 30 minutes of ghrelin administration. Treatment is maintained for up to 21 days of a 28-day cycle. Optionally, the treatment continues in 28-day cycles until disease progression or unacceptable toxicity. Treatment is reduced or discontinued for severe adverse reaction, including hepatotoxicity (see, e.g., STIVARGA (regorafenib) Label, accessible at www.accessdatafda.gov/drugsatfda_docs/label/2013/204369lbl.pdf).

It is expected that patients administered regorafenib in combination with ghrelin will have less toxicity (e.g., hepatotoxicity) compared to patients treated with regorafenib alone. It is further expected that the daily dose, duration of treatment (e.g., number of days in a 28-day cycle and/or number of cycles) of regorafenib are increased in the patients who receive ghrelin.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

1. A method for increasing survival in a subject having cancer, the method comprising administering to the subject ghrelin or a ghrelin variant, and a chemotherapeutic agent, thereby increasing survival or progression-free survival of the subject.

2. A method for increasing dose intensity, dose frequency, and/or duration of treatment of a chemotherapeutic agent in a subject having cancer, the method comprising administering to the subject ghrelin or a ghrelin variant, and the chemotherapeutic agent such that the dose intensity, dose frequency, and/or duration of treatment of the chemotherapeutic agent is increased.

3.-4. (canceled)

5. The method of claim 2, wherein ghrelin is administered.

6. The method of claim 2, wherein a ghrelin variant is administered.

7. The method of claim 2, wherein the chemotherapeutic agent is a kinase inhibitor, an anti-cancer antibody, an alkylating agent, an alkaloid, an antitumor antibiotic, an antimetabolite, or a topoisomerase inhibitor.

8. The method of claim 2, wherein the chemotherapeutic agent is a platinum-based antineoplastic drug (platin) or a kinase inhibitor.

9. The method of claim 8, wherein the chemotherapeutic agent is cisplatin.

10. The method of claim 8, wherein the chemotherapeutic agent is regorafenib.

11. The method of claim 2, wherein ghrelin or the ghrelin variant is administered after the subject has consumed a meal.

12. The method of claim 11, wherein the ghrelin or ghrelin variant is administered within about 2 hours of consumption of the meal.

13. The method of claim 11, wherein the meal is a low-fat meal.

14. The method of claim 13, wherein the meal contains less than about 30% fat.

15. The method of claim 11, wherein the chemotherapeutic agent is administered after administration of ghrelin or the ghrelin variant.

16. The method of claim 15, wherein the chemotherapeutic agent is administered within about 2 hours of consumption of the meal.

17. The method of claim 15, wherein the chemotherapeutic agent is administered within about 2 hours of ghrelin or ghrelin variant administration.

18. The method of claim 2, wherein the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, LY444711, MK-0677, L-692,429, NNC 26-0703, EP 1572, Capromorelin (CP-424, 391-18, RQ-00000005), L-252,564, NN703, G-7203, S-37435, SM-130868, EX-1314, ulimorelin, macimorelin (acetate), anamorelin, ipamorelin, PF-5190457, AMX-213, tabimorelin, capromorelin, GHRP-6, or a combination thereof.

19. The method of claim 2, wherein the cancer is colorectal cancer, colon cancer, rectal cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, leukemia, acute leukemias, acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma or retinoblastoma.

20.-21. (canceled)

22. A method for increasing dose intensity, frequency, and/or duration of treatment with a chemotherapeutic agent in a subject having cancer, the method comprising administering to the subject ghrelin or a ghrelin variant and the chemotherapy agent, wherein the chemotherapy agent is a kinase inhibitor or a platinum-based antineoplastic drug (platin), wherein the ghrelin or ghrelin variant is administered with or after a meal.

23.-50. (canceled)
